(19) United States
(12) Patent Application Publication
(10) Pub. No.: US 2026/0174341 A1
(43) Pub. Date: Jun. 25, 2026

(54) BIOLOGICAL INFORMATION MEASUREMENT DEVICE, BIOLOGICAL INFORMATION MEASUREMENT SYSTEM, AND CHARGER

(71) Applicant: OMRON HEALTHCARE Co., Ltd., Kyoto (JP)

(72) Inventors: Moe KATO, Kyoto (JP); Nobuaki YOSHIMURA, Kyoto (JP); Shuhei MUNEHIRA, Kyoto (JP); Takaaki OKANISHI, Kyoto (JP); Yoshihide TOKKO, Kyoto (JP); Jun NAKATSUKA, Kyoto (JP); Kosuke ABE, Kyoto (JP)

(21) Appl. No.: 19/545,631

(22) Filed: Feb. 20, 2026

Related U.S. Application Data
(63) Continuation of application No. PCT/JP2024/040872, filed on Nov. 18, 2024.

(30) Foreign Application Priority Data
Jan. 31, 2024 (JP) 2024-013767

(51) Int. Cl. A61B 5/0205; A61B 5/00; A61B 5/021; A61B 5/022; A61B 5/282; A61B 5/332

(52) U.S. Cl. CPC A61B 5/0205; A61B 5/02141; A61B 5/02225; A61B 5/282; A61B 5/332; A61B 5/681; A61B 5/742; A61B 2560/0219; A61B 2560/0468

(57) ABSTRACT

To increase a withstand voltage between a biological information measuring electrode and a charging terminal without increasing a biological information measurement device in size. A biological information measurement device includes: an electrocardiogram measuring unit configured to measure an electrocardiograph waveform of a subject and including an electrode; a body portion including a bottom portion configured to face and contact an arm portion of the subject in an attachment state; a charging terminal that receives power for charging; and an insulating member configured to separate the charging terminal from the electrode.

### TECHNICAL FIELD

This application is the U.S. national stage application filed pursuant to 35 U.S.C. 365(c) and 120 as a continuation of International Patent Application No. PCT/JP2024/040872, filed Nov. 18, 2024, which application claims priority to Japanese Patent Application No. 2024-013767, filed Jan. 31, 2024, which applications are incorporated herein by reference in their entireties.

### FIELD

The present invention relates to a biological information measurement device used by being attached to a human body, a biological information measurement system including the biological information measurement device, and a charger used for charging the biological information measurement device.

### BACKGROUND ART

In recent years, it has become common for individuals to measure their own physical and health information such as electrocardiograph signals and blood pressure values (hereinafter also referred to as biological information), on a daily basis using measurement apparatuses, and to utilize measurement results for health management. For this reason, a demand for devices where importance is placed on portability has been increasing, and portable measurement devices attached to a wrist have been proposed (for example, Patent Document 1).

Patent Document 1 discloses a wearable device that is capable of measuring biological information such as an electrocardiograph signal by being attached to a wrist of a human body. At a rear surface of a body of the wearable device, a biological information measuring electrode for measuring the biological information and a charging terminal for charging a built-in battery are disposed.

In a small biological information measurement device such as a wearable device, when a biological information measuring electrode and a charging terminal are disposed at the same surface, a withstand voltage between the biological information measuring electrode and the charging terminal is lowered.

On the other hand, when a distance between the biological information measuring electrode and the charging terminal is increased in order to increase the withstand voltage, the biological information measurement device is increased in size.

### CITATION LIST

### Patent Document

Patent Document 1: WO 2023/036073

### SUMMARY OF INVENTION

### Technical Problem

In view of the existing technology as described above, an object of the present invention is to provide a technology capable of increasing a withstand voltage between a biological information measuring electrode and a charging terminal without increasing a biological information measurement device in size.

### Solution to Problem

To solve the above problems, an embodiment of the present invention is a biological information measurement device including an electrocardiogram measuring unit configured to measure an electrocardiographic waveform of a subject and including an electrode, a body portion including a bottom portion configured to face and contact an arm portion of the subject in an attachment state, a charging terminal that receives power for charging, and an insulating member configured to separate the charging terminal from the electrode.

In this way, the electrode for measuring the electrocardiographic waveform, which is the biological information measuring electrode, and the charging terminal are separated by the insulating member, and thus it is not necessary to increase a distance between the biological information measuring electrode and the charging terminal, and thus it is possible to increase a withstand voltage between the biological information measuring electrode and the charging terminal without increasing the biological information measurement device in size.

In an embodiment of the present invention, the insulating member may cover the charging terminal at a side facing the arm portion, the charging terminal being provided at the bottom portion.

This can further enhance insulation properties between the biological information measuring electrode and the charging terminal and thus further increase a withstand voltage between the biological information measuring electrode and the charging terminal.

In an embodiment of the present invention, a bottom cover disposed at the bottom portion and configured to cover a region other than the electrode may be provided, wherein the insulating member may constitute at least a part of the bottom cover.

This can increase, by the bottom cover of the body portion of the biological information measurement device, the withstand voltage between the biological information measuring electrode and the charging terminal without increasing the biological information measurement device in size.

In an embodiment of the present invention, the insulating member may include an openable and closable insertion portion into which a charger-side terminal to be electrically connected to the charging terminal is inserted.

This allows simplicity of charging operation not to be impaired by the insulating member capable of increasing the withstand voltage between the biological information measuring electrode and the charging terminal without increasing the biological information measurement device in size.

In an embodiment of the present invention, the insulating member may have elasticity, and between the charging terminal and the insulating member covering the charging terminal at the side facing the arm portion, a space that allows the insulating member to deform toward the charging terminal by insertion of the charger-side terminal may be formed.

This allows the insulating member to deform according to the insertion operation of the charger-side terminal, even when the insertion operation of the charger-side terminal is taken into consideration, maintaining the withstand voltage between the biological information measuring electrode and the charging terminal.

In an embodiment of the present invention, a blood pressure measuring unit configured to measure a blood pressure of the subject and including a cuff may be provided, wherein the bottom cover may cover at least a part of the cuff.

This can increase, even in the biological information measurement device that measures a blood pressure as the biological information, the withstand voltage between the biological information measuring electrode and the charging terminal without increasing the biological information measurement device in size.

In an embodiment of the present invention is a biological information measurement system including a biological information measurement device configured to measure biological information and a charger for charging the biological information measurement device, the biological information measurement device including an electrocardiogram measuring unit configured to measure an electrocardiographic waveform of a subject and including an electrode, a body portion including a bottom portion configured to face and contact an arm portion of the subject in an attachment state and a front surface portion located on a side opposite to the bottom portion, a charging terminal that is provided at the bottom portion and receives power for charging, and an insulating member configured to cover the charging terminal at a side facing the arm portion and including an openable and closable insertion portion into which a charger-side terminal to be electrically connected to the charging terminal is inserted, the charger including a bottom portion support portion provided with the charger-side terminal, a front surface support portion configured to support the front surface portion of the biological information measurement device in which the charger-side terminal is electrically connected to the charging terminal and to position the body portion of the biological information measurement device with respect to the charger-side terminal, and a connection portion configured to openably and closably connect the front surface support portion to the bottom portion support portion, the charger being configured to electrically connect the charging terminal to the charger-side terminal by closing the front surface support portion supporting the front surface portion of the biological information measurement device with respect to the bottom portion support portion.

Thus, since the electrode for measuring the electrocardiographic waveform, which is the biological information measuring electrode, is separated from the charging terminal by the insulating member, a distance between the biological information measuring electrode and the charging terminal does not need to be increased, and therefore, the biological information measurement system can be configured with the biological information measurement device capable of increasing the withstand voltage between the biological information measuring electrode and the charging terminal without an increase in size, and the charger capable of simply and reliably charging the biological information measurement device.

In an embodiment of the present invention, the front surface portion may include a display unit capable of displaying information, the body portion may include a side surface portion between the front surface portion and the bottom portion the front surface support portion may include a sidewall portion that comes into contact with the side surface portion including both sides sandwiching the body portion and one side orthogonal to both the sides, and a bottom wall portion configured to cover at least a part of the display unit.

This can appropriately position the biological information measurement device with respect to the charger and check various kinds of information such as charging progress information by the display unit that is partially exposed.

In an embodiment of the present invention, the charger-side terminal may be provided at the bottom portion support portion so as to be spring-stretchably in a direction of connection with the charging terminal.

This can bring the charger-side terminal into pressure contact with the charging terminal by spring elasticity, electrically connecting the charger and the biological information measurement device more reliably.

In an embodiment of the present invention is a charger used for charging a biological information measurement device including an electrocardiogram measuring unit configured to measure an electrocardiographic waveform of a subject and including an electrode, a body portion including a bottom portion configured to face and contact an arm portion of the subject in an attachment state and a front surface portion located on a side opposite to the bottom portion, a charging terminal that is provided at the bottom portion and receives power for charging, and an insulating member configured to cover the charging terminal at a side facing the arm portion and including an openable and closable insertion portion into which a charger-side terminal to be electrically connected to the charging terminal is inserted, the charger including a bottom portion support portion provided with the charger-side terminal, a front surface support portion configured to support the front surface portion of the biological information measurement device in which the charger-side terminal is electrically connected to the charging terminal and to position the body portion of the biological information measurement device with respect to the charger-side terminal, and a connection portion configured to openably and closably connect the front surface support portion to the bottom portion support portion, the charger being configured to electrically connect the charging terminal to the charger-side terminal by closing the front surface support portion supporting the front surface portion of the biological information measurement device with respect to the bottom portion support portion.

This eliminates, by separating the electrode for measuring the electrocardiographic waveform, which is the biological information measuring electrode, and the charging terminal by the insulating member, the need of increasing a distance between the biological information measuring electrode and the charging terminal, thus providing the charger capable of simply and reliably charging the biological information measurement device capable of increasing the withstand voltage between the biological information measuring electrode and the charging terminal without being increased in size.

### Advantageous Effects of Invention

An embodiment of the present invention can increase a withstand voltage between a biological information measuring electrode and a charging terminal without increasing a biological information measurement device in size.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is an appearance perspective view illustrating an outline of a biological information measurement device according to an example of the present invention.

FIG. 2 is a side view illustrating the outline of the biological information measurement device according to the example.

FIG. 3 is an explanatory diagram illustrating a disposition relationship when the biological information measurement device according to the example is attached to a wrist.

FIG. 4 is an appearance view when a body portion of the biological information measurement device according to the example is viewed from a bottom portion side.

FIG. 5 is an appearance view when a main body case of the biological information measurement device according to the example from which a cuff cover is removed is viewed from a bottom surface side.

FIG. 6 is an appearance view when the cuff cover of the biological information measurement device according to the example is viewed from the bottom portion side.

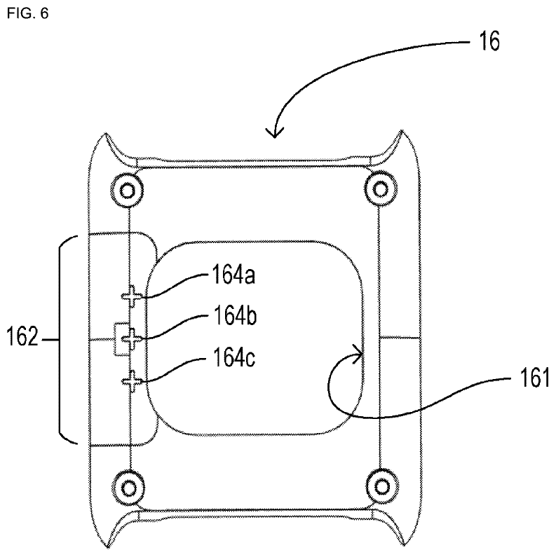

FIG. 7A and FIG. 7B are perspective views when the cuff cover of the biological information measurement device according to the example is viewed from an inner surface side.

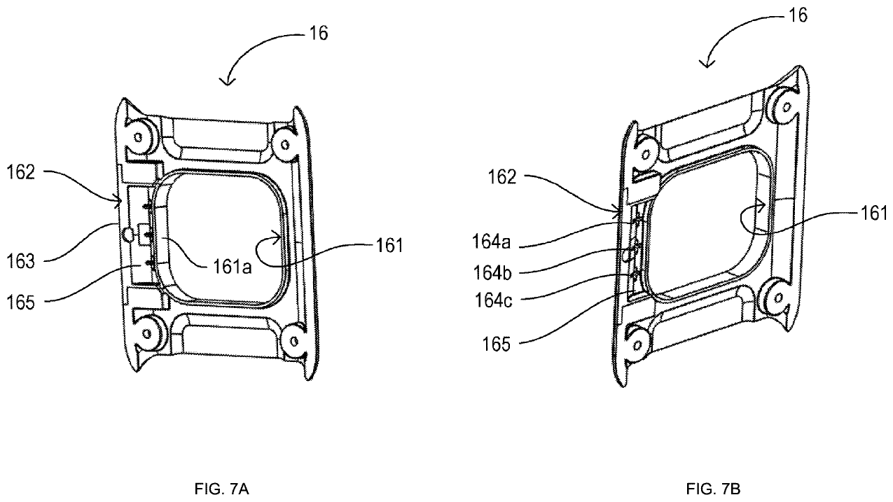

FIG. 8 is a block diagram illustrating a functional configuration of a biological information measurement system including the biological information measurement device and a charger according to the example.

FIG. 9A and FIG. 9B are a side view and an appearance perspective view of the charger according to the example.

FIG. 10A is an appearance perspective view illustrating a state of the charger according to the example before the biological information measurement device is connected, and FIG. 10B is an appearance perspective view illustrating a state where the biological information measurement device is connected to the charger according to the example.

### DESCRIPTION OF EMBODIMENTS

Embodiments of the present invention will be specifically described below with reference to the drawings.

### Example 1

Hereinafter, an example of the embodiments of the present invention will be described. It should be noted that the dimension, material, shape, relative arrangement, and the like of the components described in the present examples are not intended to limit the scope of this invention to them alone, unless otherwise stated.

### Device Configuration

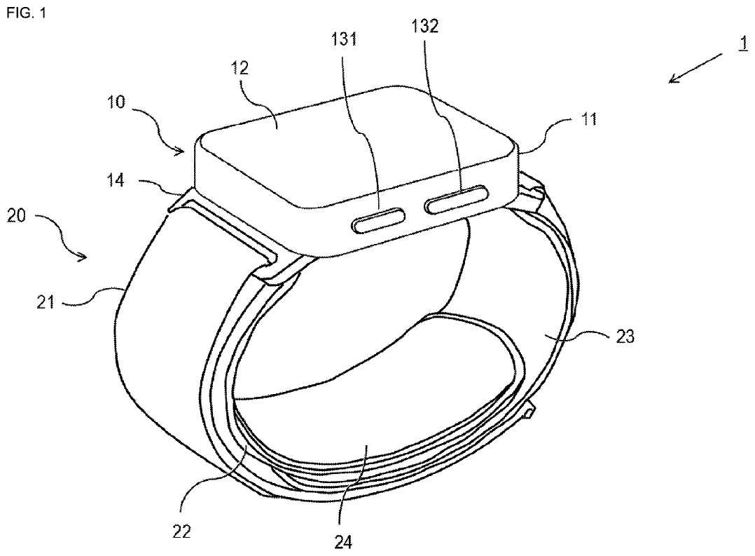

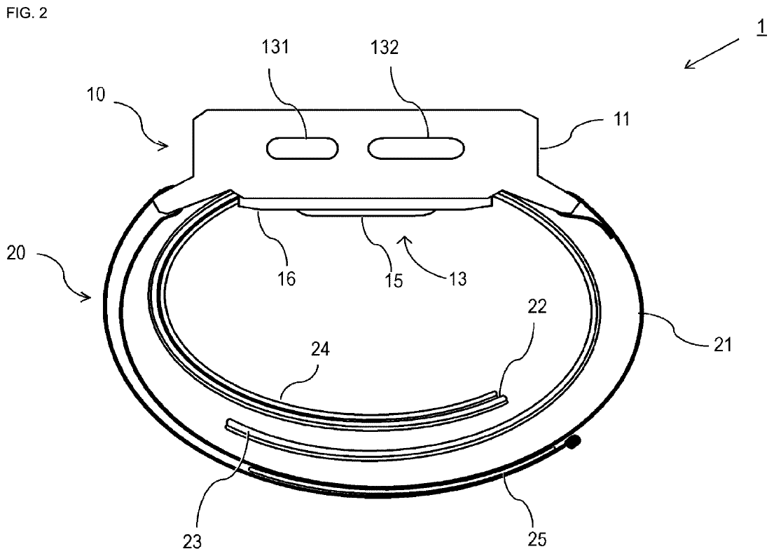

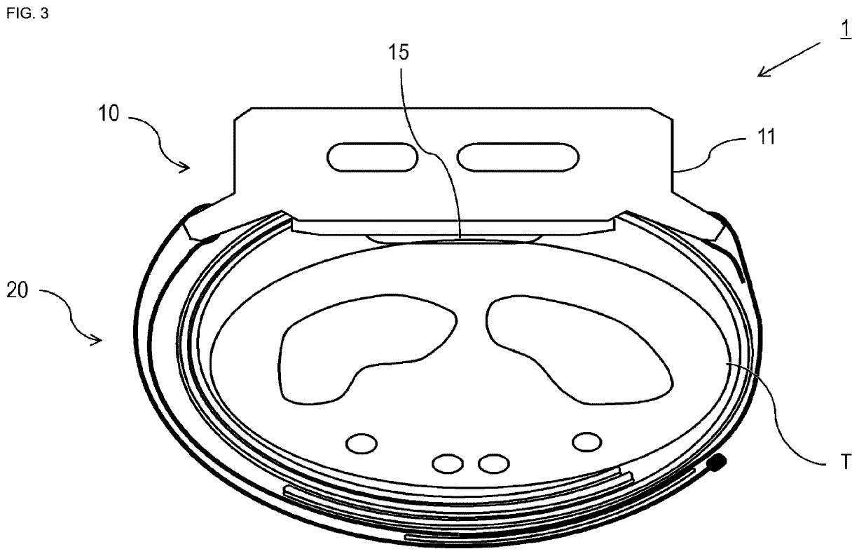

FIG. 1 is an appearance perspective view illustrating an outline of a configuration of a biological information measurement device 1 according to the present example. FIG. 2 is a side view illustrating the outline of the configuration of the biological information measurement device 1 according to the present example. As illustrated in FIG. 1 and FIG. 2, the biological information measurement device 1 is schematically a wristwatch-type wearable device including a body portion 10 and a belt portion 20, and is capable of measuring biological information such as a pulse wave (pulse), a blood pressure value, and an electrocardiographic waveform while being attached to a wrist T of a subject. FIG. 3 illustrates a disposition relationship between the wrist and configurations of the biological information measurement device 1 according to the present example when the biological information measurement device 1 is attached to the wrist T.

As illustrated in FIG. 1 and FIG. 2, the body portion 10 includes a body case 11 and a cuff cover 16 described below. The main body case 11 is provided with a display 12 (for example, an organic EL display or the like), operation buttons 131 and 132, a lug 14, and the like, and is also provided with a sensor housing portion 15 in which various sensors described below are housed. Note that, in the present example, a side on which the display 12 is formed is referred to as a front surface of the main body case 11, and a side on which the sensor housing portion 15 is formed is referred to as a bottom surface 11a of the main body case 11. Note that, in the present example, the operation buttons 131 and 132 are formed of a conductor and also function as electrodes for electrocardiographic waveform measurement. The bottom surface 11a of the body case 11, and a resin cover and the cuff cover 16 covering the bottom surface 11a constitute a bottom portion 13 of the body portion 10. The display 12 corresponds to the front surface portion and the display unit of an embodiment of the present invention.

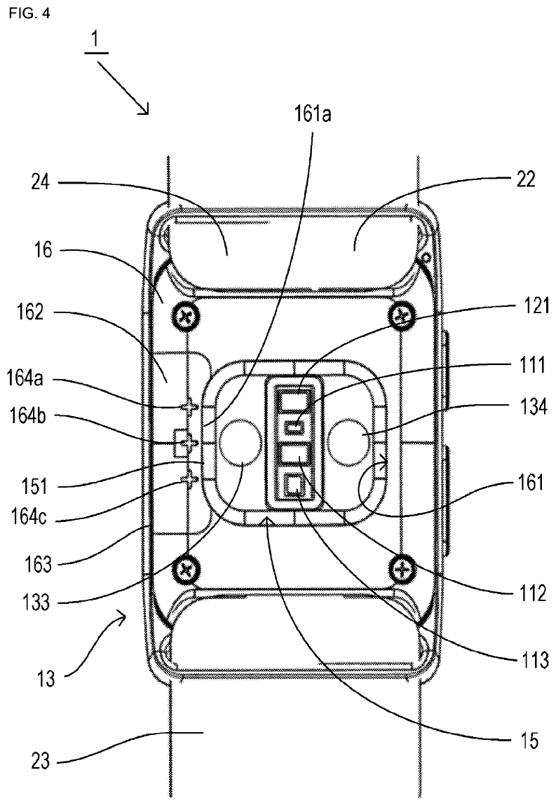

FIG. 4 is an appearance view of the body portion 10 when viewed from the bottom portion 13 side. As illustrated in FIG. 4, at the bottom portion 13 of the body portion 10, a region of a central portion covered with the light-transmissive resin cover, and a region that corresponds to an outer circumference of the region of the central portion and that is covered with the cuff cover 16 are present. The sensor housing portion 15 is located in the region of the central portion of the main body case 11 in plan view and is covered with the light-transmissive resin cover at the bottom portion 13. Moreover, as illustrated in FIG. 2 and FIG. 3, the sensor housing portion 15 is formed so as to protrude toward the wrist T relative to the cuff cover 16 in an attachment state.

A first electrode 133 and a second electrode 134 are provided at the sensor housing portion 15 of the bottom surface 11a of the main body case 11 such that contact surfaces with a human body are exposed. One of the first electrode 133 and the second electrode 134 functions as a GND electrode during electrocardiographic waveform measurement. During the electrocardiographic waveform measurement, the biological information measurement device 1 is attached, the contact surfaces of the first electrode 133 and the second electrode 134 are brought into contact with a skin surface at an attachment portion, and the operation button 131 or 132 is touched with a finger on a side where the biological information measurement device 1 is not attached. Thus, the electrocardiographic waveform measurement can be performed by I induction. The first electrode 133 and the second electrode 134 correspond to the electrodes of an embodiment of the present invention.

A first LED 111, a second LED 113, a first photodiode (PD) 112, and a second PD 121 can be seen through the resin cover from the bottom portion 13 side of the body portion 10. Note that, in the present example, the first LED 111 emits green irradiation light, and the second LED 113 emits red light and/or infrared light in addition to green light.

On the bottom portion 13 side of the body portion 10, as described above, the sensor housing portion 15 is exposed from a rectangular opening portion 161 formed in a central portion of the cuff cover 16. Then, in the cuff cover 16, a charging terminal covering portion 162 is provided adjacent to a longitudinal direction end portion 151 of the sensor housing portion 15. The charging terminal covering portion 162 is formed from an edge portion 161a of the opening portion 161 adjacent to the longitudinal direction end portion 151 of the sensor housing portion 15 to a lateral direction end portion 163 of the cuff cover 16. The charging terminal covering portion 162 has three charging pin press-fit holes 164a to 164c formed along a longitudinal direction of the main body case 11. The charging pin press-fit holes 164a to 164c are openable and closable holes for press-fitting charging pins 240a to 240c provided at a charger 2 described below. The cuff cover 16 corresponds to the bottom cover of an embodiment of the present invention. The charging pin press-fit holes 164a to 164c correspond to the insertion portions of an embodiment of the present invention. The charging pins 240a to 240c correspond to the charger-side terminals of an embodiment of the present invention.

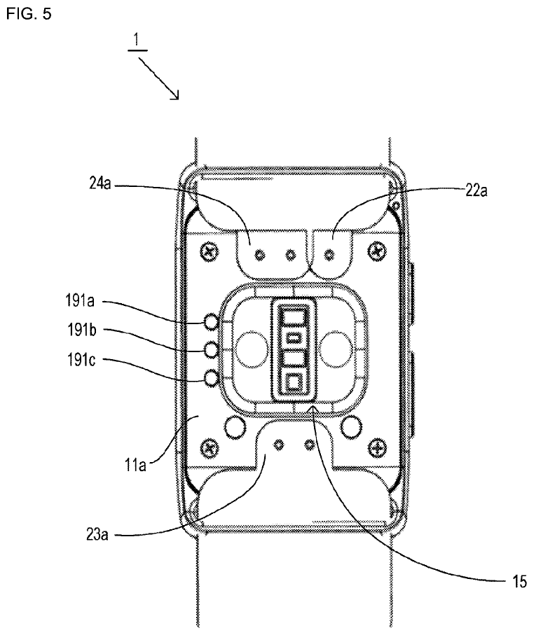

FIG. 5 illustrates an appearance view of the main body case 11 from which the cuff cover 16 is removed when viewed from the bottom surface 11a side. As illustrated in FIG. 5, at the bottom surface 11a of the main body case 11 side facing the charging terminal covering portion 162, charging terminals 191a to 191c are disposed along the longitudinal direction outside the longitudinal direction end portion 151 of the sensor housing portion 15. The three charging pin press-fit holes 164a to 164c of the charging terminal covering portion 162 are formed at positions corresponding to the charging terminals 191a to 191c in a thickness direction of the body portion 10. A space 165 that allows the charging terminal covering portion 162 to deform toward the charging terminals 191a to 191c is provided between the charging terminal covering portion 162 and the charging terminals 191a to 191c. In this manner, the charging terminals 191a to 191c are separated from the first electrode 133 and the second electrode 134 by the charging terminal covering portion 162. With such a configuration, it is not necessary to increase a distance between the first electrode 133 and the second electrode 134, and the charging terminals 191a to 191c, and thus, it is possible to increase a withstand voltage between the first electrode 133 and the second electrode 134, and the charging terminals 191a to 191c without increasing the biological information measurement device 1 in size.

By pressing the charging pins 240a to 240c against the charging pin press-fit holes 164a to 164c, the charging terminal covering portion 162 is elastically deformed and recessed toward the charging terminals 191a to 191c, the charging pin press-fit holes 164a to 164c are pushed and opened, and the charging pins 240a to 240c are press-fitted into the charging pin press-fit holes 164a to 164c. When the charging pins 240a to 240c are further press-fitted into the charging pin press-fit holes 164a to 164c, end portions of the charging pins 240a to 240c abut against the charging terminals 191a to 191c, and the charging pins 240a to 240c are electrically connected to the charging terminals 191a to 191c. As described below, the charging pins 240a to 240c have spring elasticity and are provided at the charger 2, and therefore, by the charging pins 240a to 240c being brought into pressure contact with the charging terminals 191a to 191c, the electrical connection can be ensured. The charging pin press-fit holes 164a to 164c are formed so that a user does not touch the charging terminals 191a to 191c from the charging pin press-fit holes 164a to 164c only by touching the charging terminal covering portion 162 from the bottom portion 13 side. The cuff cover 16 can be manufactured by, for example, two-color molding in which the charging terminal covering portion 162 is formed of a member made of non-conductive (insulating) and elastic elastomer, and portions other than the charging terminal covering portion 162 are formed of a hard resin. The portions of the cuff cover 16 other than the charging terminal covering portion 162 may also be formed of non-conductive (insulating) members, or may be formed of elastic members. The entire cuff cover 16 including the charging terminal covering portion 162 may be formed of a member made of elastomer. The body case 11 and the bottom surface 11a correspond to the body portion and the bottom portion of an embodiment of the present invention, respectively. The charging terminals 191a to 191c correspond to the charging terminals of an embodiment of the present invention. The charging terminal covering portion 162 corresponds to the insulating member of an embodiment of the present invention. The space 165 corresponds to the space of an embodiment of the present invention.

With such a charger 2, the biological information measurement device 1 can be charged easily and reliably. Such biological information measurement device 1 and charger 2 can constitute a biological information measurement system 3 according to the biological information measurement device 1 capable of increasing the withstand voltage between the first electrode 133 and the second electrode 134, and the charging terminals 191a to 191c without an increase in size, and the charger 2 capable of easily and reliably charging the biological information measurement device 1.

The belt portion 20 includes a belt 21 and a surface fastener 25 for fixing the biological information measurement device 1 to the wrist T, and also includes a first pressing cuff 22 and a second pressing cuff 23 for compressing an artery in the wrist T, and a sensing cuff 24 for detecting a pressure pulse wave. As illustrated in FIG. 5, at the bottom surface 11a of the main body case 11, in a region where the sensor housing portion 15 is not provided in plan view, a first connection portion 22a and a second connection portion 24a that connect the main body case 11 to the first pressing cuff 22 and the sensing cuff 24, respectively, and a third connection portion 23a that similarly connects the main body case 11 to the second pressing cuff 23 are provided. The first connection portion 22a, the second connection portion 24a, and the third connection portion 23a are covered with the cuff cover 16. The cuff cover 16 protects the first connection portion 22a, the second connection portion 24a, and the third connection portion 23a, and also has a function to fix the respective cuffs 22, 23, and 24 to the main body case 11. The first pressing cuff 22 is provided at the body portion on one side in a circumferential direction, and the second pressing cuff 23 is provided at the body portion 10 on another side in the circumferential direction. The first pressing cuff 22, the second pressing cuff 23, and the sensing cuff 24 correspond to the cuffs of an embodiment of the present invention. The first connection portion 22a, the second connection portion 24a, and the third connection portion 23a correspond to at least some of the cuffs of an embodiment of the present invention. The wrist T corresponds to the arm portion of an embodiment of the present invention.

A rechargeable battery 193, a control board (not illustrated), a piezoelectric pump 141, a valve 142, a pressure sensor 143, a flow path plate 144, and the like are housed inside the main body case 11.

### Functional Configuration of Biological Information Measurement Device and Charger

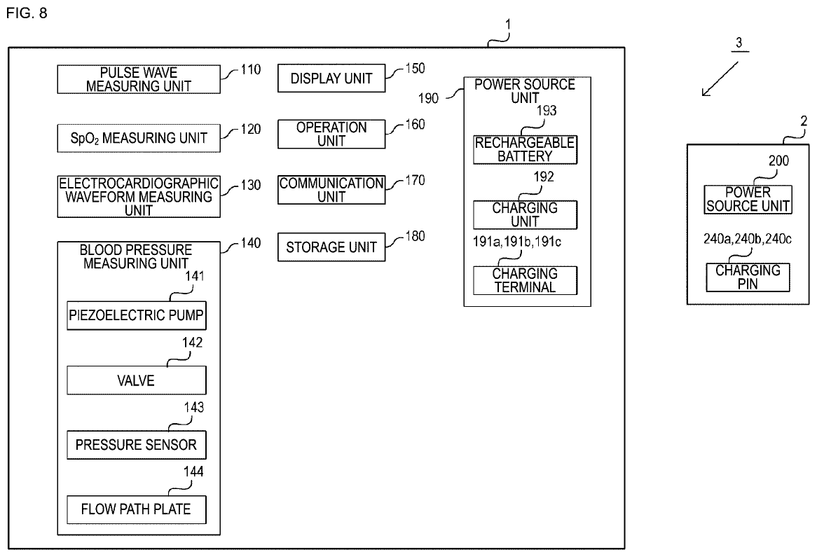

Next, a functional configuration of the biological information measurement system 3 including the biological information measurement device 1 and the charger 2 will be described. FIG. 8 is a block diagram illustrating the functional configuration of the biological information measurement device 1 and the charger 2. As illustrated in FIG. 8, the biological information measuring device 1 according to the present example includes functional units of a pulse wave measuring unit 110, a blood oxygen saturation (SpO₂) measuring unit 120, an electrocardiographic waveform measuring unit 130, a blood pressure measuring unit 140, a display unit 150, an operation unit 160, a communication unit 170, a storage unit 180, and a power source unit 190. A processor such as a CPU provided at the body portion 10 reads a program from a memory such as a RAM and executes the program to control each configuration of the biological information measurement device 1, thereby implementing these functional units. The charger 2 includes a power source unit 200 that supplies power via the charging pins 240a to 240c, and the charging pins 240a to 240c, which will be described below. The power source unit 200 includes, for example, an AC/DC converter. The power source unit 200 may be further connected to a commercial power system and receive supply of AC power from the commercial power system. The charger 2 corresponds to the charger of an embodiment of the present invention.

The pulse wave measuring unit 110 includes the first LED 111, the second LED 113, and the first PD 112, and measures a pulse wave by a so-called photoplethysmographic method to calculate a pulse. Specifically, green light is emitted from the first LED 111 and the second LED 113, and reflected light reflected in a living body is received by the first PD 112, so that a blood flow volume that changes with a pulsation of a heart (a change in volume of a blood vessel) is detected, and a pulse wave is measured. The pulse wave is an example of the biological information, and the pulse wave measuring unit 110 is an example of a biological information measuring unit.

An SpO₂ measuring unit 120 includes the second LED 113 and the second PD 121, and the second PD 121 receives reflection light of red light or infrared light emitted from the second LED 113, so that the SpO₂ measuring unit 120 measures blood oxygen saturation from an intensity of the reflection light. The blood oxygen saturation is an example of the biological information, and the SpO₂ measuring unit 120 is an example of the biological information measuring unit.

The electrocardiographic waveform measuring unit 130 includes the operation buttons 131 and 132, the first electrode 133, the second electrode 134 provided at the bottom portion 13 of the body portion 10, and an electrocardiographic waveform measuring circuit (not illustrated), and measures an electrocardiographic waveform by a so-called I induction method. Specifically, an electrocardiographic waveform is measured based on a potential difference between the first electrode 133 and the second electrode 134 that are in contact with the wrist T of one arm in the attachment state, and a finger of another hand in contact with the operation button 131 or 132 functioning as the electrode. The electrocardiographic waveform is an example of the biological information, and the electrocardiographic waveform measuring unit 130 corresponds to the electrocardiogram measuring unit of an embodiment of the present invention.

The blood pressure measuring unit 140 includes the piezoelectric pump 141, the valve 142, the pressure sensor 143, the flow path plate 144, and the above-described first pressing cuff 22, second pressing cuff 23, and sensing cuff 24, and measures a blood pressure by a so-called oscillometric method. The flow path plate 144 is a conductive member (metal), and flow paths for feeding gas from the piezoelectric pump 141 to the respective cuffs 22, 23, and 24 are formed therein. The blood pressure measurement using the oscillometric method is a known technique, and therefore the detailed description thereof is omitted. The blood pressure is an example of the biological information, and the blood pressure measuring unit 140 corresponds to the blood pressure measuring unit of an embodiment of the present invention.

The display unit 150 includes the display 12, and displays various types of information such as a measurement result of the biological information and a menu screen. The operation unit 160 includes the operation buttons 131 and 132, and receives an input operation by the user via these buttons. The communication unit 170 includes an antenna for wireless communication (not illustrated), and performs information communication with another electronic device such as an information processing terminal by, for example, BLE communication. Note that a terminal for wired communication may be provided.

The storage unit 180 includes a main storage device (not illustrated) such as a random access memory (RAM) and stores various types of information such as application programs and the measured biological information. S long-term storage medium such as a flash memory in addition to the RAM, for example, may be provided.

The power source unit 190 includes the charging terminals 191a to 191c, a charging unit 192, and a rechargeable battery 193, and functions as a power supply source to each unit constituting the biological information measurement device 1. The charging unit 192 is a circuit that rectifies power input from the charging terminals 191a to 191c, and performs conversion into a predetermined current and/or voltage for charging the rechargeable battery 193, for example. As the rechargeable battery 193, a general-purpose rechargeable battery such as a lithium ion battery can be adopted, and the rechargeable battery 193 can be repeatedly charged by receiving supply of power via the charging terminals 191a to 191c.

### Configuration of Charger

The charger 2 according to the present example will be described below.

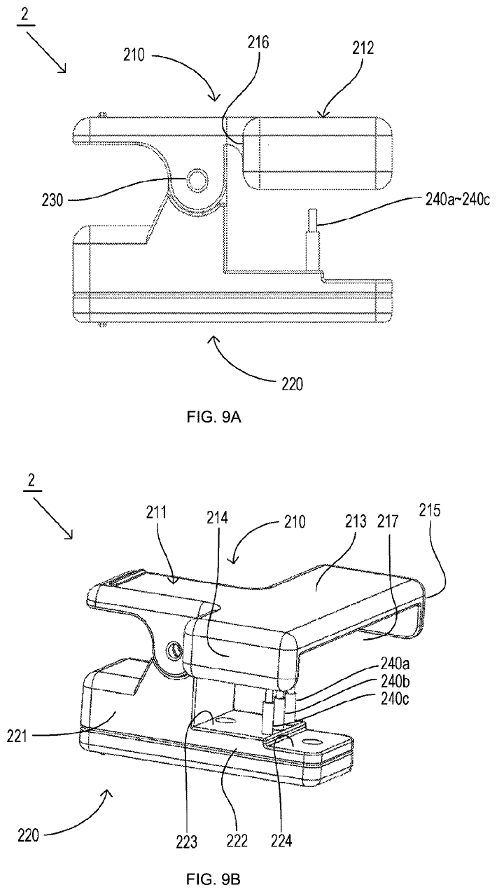

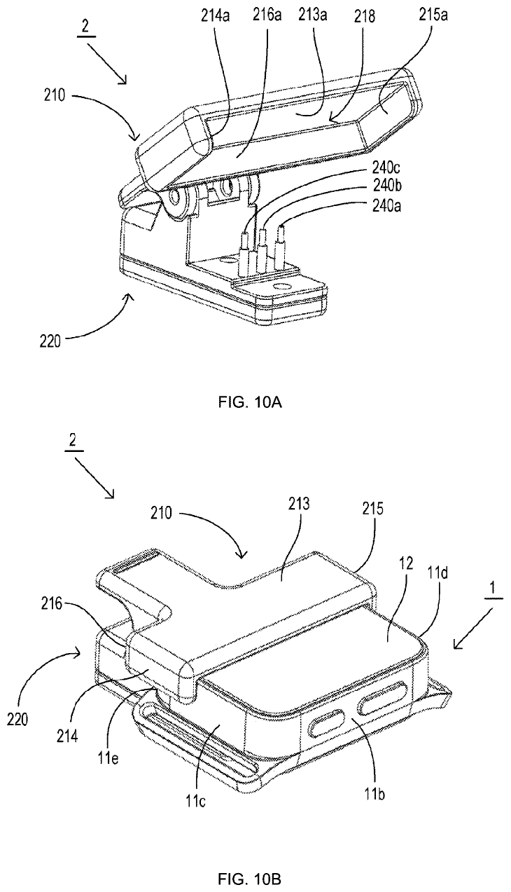

FIG. 9A is a side view of the charger 2, and FIG. 9B is a perspective view of the charger 2. FIG. 10A is a perspective view of the charger 2 in a state where the biological information measurement device 1 is about to be connected, and FIG. 10B is a perspective view of the charger 2 in a state where the biological information measurement device 1 is connected.

The charger 2 is a clip-shaped structural body in which a positioning portion 210 having a substantially T-shape in plan view is attached to a base portion 220 provided with the charging pins 240a to 240c so as to be rotatable about a shaft portion 230. The positioning portion 210 and the base portion 220 are formed of, for example, resin such as polycarbonate or ABS, and the charging pins 240a to 240c are formed of conductive members. The positioning portion 210, the base portion 220, and the shaft portion 230 correspond to the front surface support portion, the bottom portion support portion, and the connection portion of an embodiment of the present invention, respectively.

The positioning portion 210 includes a support portion 211 supported by the shaft portion 230, and a holding portion 212 extending from an end portion of the support portion 211 in parallel to the shaft portion 230. The holding portion 212 has a substantially rectangular parallelepiped box shape in which an end portion on an opposite side to the support portion 211 and a portion facing the base portion 220 are opened. The holding portion 212 includes an upper wall portion 213 having a substantially rectangular plate shape that faces the base portion 220 and extends in parallel to the shaft portion 230, a lateral wall portion 214 and a lateral wall portion 215 having a substantially rectangular plate shape that extend from longitudinal direction end portions of the upper wall portion 213 toward the base portion 220, and a back wall portion 216 having a substantially rectangular plate shape that extends from an end portion of the upper wall portion 213 on the support portion 211 side toward the base portion 220 and is orthogonal to the lateral wall portion 214 and the lateral wall portion 215. An opening portion 217 is formed at end portions of the upper wall portion 213, the lateral wall portion 214, and the lateral wall portion 215 on an opposite side of the support portion 211. The lateral wall portion 214, the lateral wall portion 215, and the back wall portion 216 correspond to the sidewall portions of an embodiment of the present invention. The upper wall portion 213 corresponds to the bottom wall portion of an embodiment of the present invention.

The base portion 220 includes a shaft support portion 221 that supports the shaft portion 230, and a connection portion 222 including a surface corresponding to the positioning portion 210. At a support surface 223 of the connection portion 222 facing the positioning portion 210, the charging pins 240a to 240c are provided upright toward the upper wall portion 213. Then, the connection portion 222 is provided with a support surface 224 connected to the support surface 223 via a step. In a state where the charging pins 240a to 240c are press-fitted to a predetermined position with respect to the body portion 10 of the biological information measurement device 1, the sensor housing portion 15 is housed at the support surface 224 via the step.

When the support portion 211 of the positioning portion 210 is pushed toward the base portion 220, the holding portion 212 is opened with respect to the connection portion 222, that is, the holding portion 212 is separated from the connection portion 222, as illustrated in FIG. 10A. A space 218 that is surrounded by an upper inner wall portion 213a, a lateral inner wall portion 214a, a lateral inner wall portion 215a, and a back inner wall portion 216a, and is opened by the opening portion 217 is formed on the connection portion 222 side of the holding portion 212. The positioning portion 210 is formed in a size such that a case side surface 11e on an opposite side of a case side surface 11b on which the operation buttons 131 and 132 of the main body case 11 of the biological information measurement device 1 are disposed abuts against the back inner wall portion 216a, and the case side surface 11c and the case side surface 11d on the case side surface 11e side sandwiching the case side surface 11e partially abut against the lateral inner wall portion 214a and the lateral inner wall portion 215a, respectively. The upper wall portion 213 of the holding portion 212 is formed to have a size that covers substantially half of the display 12 in the lateral direction in a state where the case side surface 11e is caused to abut against the back inner wall portion 216a, and the case side surface 11c and the case side surface 11d on the case side surface side 11e sandwiching the case side surface 11e are caused to partially abut against the lateral inner wall portion 214a and the lateral inner wall portion 215a, respectively.

In a state where the holding portion 212 of the charger 2 illustrated in FIG. 10A is opened with respect to the connection portion 222, the main body case 11 of the biological information measurement device 1 is caused to be held by the holding portion 212. To be specific, the main body case 11 is caused to be held by the holding portion 212 so that the case side surface 11e abuts against the back inner wall portion 216a, and the case side surface 11c and the case side surface 11d on the case side surface 11e side sandwiching the case side surface 11e partially abut against the lateral inner wall portion 214a and the lateral inner wall portion 215a, respectively. By causing the body case 11 to be held by the holding portion 212 in this manner, the body portion 10 of the biological information measurement device 1 is positioned with respect to the charger 2. That is, by causing the main body case 11 to be held by the holding portion 212, the charging pins 240a to 240c are positioned with respect to the charging pin press-fit holes 164a to 164c and the charging terminals 191a to 191c, respectively. Therefore, when the holding portion 212 of the charger 2 is closed with respect to the connection portion 222 in a state where the main body case 11 is caused to be held by the holding portion 212, the charging pins 240a to 240c deform the charging terminal covering portion 162 so as to be recessed toward the charging terminals 191a to 191c, and are press-fitted into the charging pin press-fit holes 164a to 164c, respectively. Further, by closing the holding portion 212 with respect to the connection portion 222, the charging pins 240a to 240c abut against the charging terminals 191a to 191c, respectively. Since the charging pins 240a to 240c are configured with spring pins spring-stretchable in a direction of connection with the charging terminals 191a to 191c, after the charging pins 240a to 240c abut against the charging terminals 191a to 191c, respectively, by closing the holding portion 212 with respect to the connection portion 222, the charging pins 240a to 240c are brought into pressure contact with the charging terminals 191a to 191c, respectively, and thus the charger 2 is electrically and reliably connected to the biological information measurement device 1. The case side surfaces 11c to 11e correspond to the side surface portions of an embodiment of the present invention. The case side surface 11c and the case side surface 11d correspond to the side surface portions on both sides sandwiching the body portion of an embodiment of the present invention, and the case side surface 11e corresponds to the surface portion on one side orthogonal to both sides sandwiching the body portion of an embodiment of the present invention.

When the rechargeable battery 193 is charged in a state where the holding portion 212 is closed to a predetermined position with respect to the connection portion 222, and the charging pins 240a to 240c are connected to the charging terminals 191a to 191c, substantially half of the display 12 is not covered with the upper wall portion 213 and is exposed. Therefore, progress of charging and the like can be displayed in a region of the display 12 exposed from the holding portion 212. A fact that the charging is completed may be displayed in the exposed region of the display 12 to notify the user of the completion of charging.

In the charger 2, a configuration is adopted in which, the biological information measurement device 1 is caused to be held by the positioning portion 210 such that about half of the main body case 11 of the biological information measurement device 1 on the case side surface 11b side on which the operation buttons 131 and 132 are disposed is exposed from the opening portion 217 of the positioning portion 210, and thus the charging pins 240a to 240c are appropriately connected to the charging terminals 191a to 191c. Therefore, it is possible to prevent the biological information measurement device 1 from being caused to be held in a wrong direction with respect to the charger 2 and to damage the charging pins 240a to 240c and the biological information measurement device 1.

### REFERENCE SIGNS LIST

1 Biological information measurement device
2 Charger
11 Body case
11a Bottom surface
Body portion
130 Electrocardiographic waveform measuring unit
133 First electrode
134 Second electrode
162 Charging terminal covering portion
191a to 191c Charging terminal
T Wrist

1. A biological information measurement device, comprising:
an electrocardiogram measuring unit configured to measure an electrocardiographic waveform of a subject and including an electrode;
a body portion including a bottom portion configured to face and contact an arm portion of the subject in an attachment state;
a charging terminal that receives power for charging;
an insulating member configured to separate the charging terminal from the electrode; and
a bottom cover disposed at the bottom portion and configured to cover a region other than the electrode, wherein
the insulating member covers the charging terminal at a side facing the arm portion, the charging terminal being provided at the bottom portion, constitutes at least a part of the bottom cover, and includes an openable and closable insertion portion into which a charger-side terminal to be electrically connected to the charging terminal is inserted.

2. The biological information measurement device according to claim 1, wherein
the insulating member has elasticity, and
between the charging terminal and the insulating member covering the charging terminal at the side facing the arm portion, a space that allows the insulating member to deform toward the charging terminal by insertion of the charger-side terminal is formed.

3. The biological information measurement device according to claim 1, comprising
a blood pressure measuring unit configured to measure a blood pressure of the subject and including a cuff, wherein
the bottom cover covers at least a part of the cuff.

4. A biological information measurement system, comprising:
a biological information measurement device configured to measure biological information; and
a charger configured to charge the biological information measurement device,
the biological information measurement device including
an electrocardiogram measuring unit configured to measure an electrocardiographic waveform of a subject and including an electrode,
a body portion including a bottom portion configured to face and contact an arm portion of the subject in an attachment state and a front surface portion located on a side opposite to the bottom portion,
a charging terminal that is provided at the bottom portion and receives power for charging, and
an insulating member configured to cover the charging terminal at a side facing the arm portion and including an openable and closable insertion portion into which a charger-side terminal to be electrically connected to the charging terminal is inserted,
the charger including
a bottom portion support portion provided with the charger-side terminal,
a front surface support portion configured to support the front surface portion of the biological information measurement device in which the charger-side terminal is electrically connected to the charging terminal and to position the body portion of the biological information measurement device with respect to the charger-side terminal, and
a connection portion configured to openably and closably connect the front surface support portion to the bottom portion support portion,
the charger being configured to electrically connect
the charging terminal to the charger-side terminal by closing the front surface support portion supporting the front surface portion of the biological information measurement device with respect to the bottom portion support portion.

5. The biological information measurement system according to claim 4, wherein
the front surface portion includes a display unit capable of displaying information,
the body portion includes a side surface portion between the front surface portion and the bottom portion,
the front surface support portion includes a sidewall portion that comes into contact with the side surface portion including both sides sandwiching the body portion and one side orthogonal to both the sides and a bottom wall portion configured to cover at least a part of the display unit.

6. The biological information measurement system according to claim 4, wherein
the charger-side terminal is provided at the bottom portion support portion so as to be spring-stretchably in a direction of connection with the charging terminal.

7. The biological information measurement system according to claim 5, wherein
the charger-side terminal is provided at the bottom portion support portion so as to be spring-stretchably in a direction of connection with the charging terminal.

8. A charger used for charging a biological information measurement device including
an electrocardiogram measuring unit configured to measure an electrocardiographic waveform of a subject and including an electrode,
a body portion including a bottom portion configured to face and contact an arm portion of the subject in an attachment state and a front surface portion located on a side opposite to the bottom portion,
a charging terminal that is provided at the bottom portion and receives power for charging, and
an insulating member configured to cover the charging terminal at a side facing the arm portion and including an openable and closable insertion portion into which a charger-side terminal to be electrically connected to the charging terminal is inserted,
the charger comprising:
a bottom portion support portion provided with the charger-side terminal;
a front surface support portion configured to support the front surface portion of the biological information measurement device in which the charger-side terminal is electrically connected to the charging terminal and to position the body portion of the biological information measurement device with respect to the charger-side terminal; and
a connection portion configured to openably and closably connect the front surface support portion to the bottom portion support portion,
the charger being configured to electrically connect
the charging terminal to the charger-side terminal by closing the front surface support portion supporting the front surface portion of the biological information measurement device with respect to the bottom portion support portion.
